(19) United States
(12) Patent Application Publication
(10) Pub. No.: US 2014/0057881 A1
(43) Pub. Date: Feb. 27, 2014

(54) Composition for the Treatment of Skin Conditions

(71) Applicant: Andrew Tuan Anh Le, Lakemba (AU)

(72) Inventor: Andrew Tuan Anh Le, Lakemba (AU)

(21) Appl. No.: 13/995,451

(22) PCT Filed: Dec. 1, 2011
(86) PCT No.: PCT/AU11/01567

§ 371 Date: Oct. 1, 2013

(30) Foreign Application Priority Data
Dec. 20, 2010 (AU) 2010905529
Dec. 24, 2010 (AU) 2010905707

(51) Int. Cl. A61K 31/58; A61K 45/06; A61K 31/137; A61K 31/352; A61K 31/573

(52) U.S. Cl. CPC A61K 31/58; A61K 31/352; A61K 31/573; A61K 31/137; A61K 45/06
USPC 514/171

(57) ABSTRACT

A topical composition for the treatment of skin conditions. The composition comprises an antifungal agent, an anti-inflammatory agent and an antimicrobial agent together with a pharmaceutically acceptable excipient. The anti-inflammatory is present at a concentration of less than 0.01% wt of the composition and the antimicrobial agent has the general formula (I): (Formula (I)).

### CROSS-REFERENCE TO RELATED APPLICATIONS

The present application claims priority from AU 2010905529 and AU 2010905707 the contents of which are incorporated herein by reference.

### FIELD

The present disclosure relates to a topical compound for the treatment of various skin conditions.

### BACKGROUND

The skin as an organ has great ability to repair itself. Optimal healing is achieved when the skin is provided with the optimal environment. Particularly, it is important that the skin cells are not disrupted through itching or scratching and that there are no bacterial or fungal infections on the skin.

To date topical steroids are the most commonly prescribed topical medications for the treatment of rash, eczema, and dermatitis. Topical steroids have anti-inflammatory properties. There are numerous side effects ranging from the severe to relatively mild associated with topical steroidal use.

For eczema other current treatments include antihistamines taken by mouth to alleviate itching, topical immunomodulators (TIMs) including tacrolimus and pimecrolimus, barrier repair creams, antibiotic creams or immunosuppressants such as cyclosporine, methotrexate or mycophenolate mofetil are used.

None of the above treat eczema or other forms of dermatitis but instead alleviate the symptoms. Furthermore, they are all associated with a large number of side effects.

There is clearly a need for a topical composition, which is both safe and effective for the treatment of skin conditions.

### SUMMARY OF THE DISCLOSURE

In one aspect, there is provide a topical composition for the treatment of a skin condition, said composition comprising:

less than 0.01% wt of at least one anti-inflammatory agent;

at least one antifungal agent;

an antimicrobial agent of the general formula:

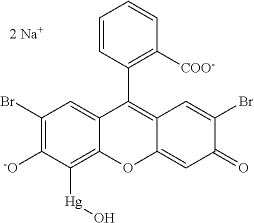

and a pharmaceutically acceptable excipient.

The anti-inflammatory agent may comprise a topical steroid. The steroid may be chosen from any one or a combination of topical steroids including but not limited to Hydrocortisone Alclometasone dipropionate, Triamcinolone acetonide, Fluocinolone acetonide, Fluticasone propionate, Hydrocortisone valerate, Hydrocortisone butyrate, Flurandrenolide, Mometasone furoate, Betamethasone dipropionate, Fluocinonide, Halcinonide, Amcinonide, Desoximetasone, Clobetasol propionate, Halobetasol proprionate, Diflorasone diacetate, Diflucortolone valerate, Hydrocortisone 17-butyrate, Methylprednisolone aceponate or Clobetasone butyrate

In one embodiment, the steroid may be less than 0.009% wt of the composition. In another embodiment, the steroid may be in the range of 0.005% wt to 0.0099% wt. In another embodiment, the steroid may be in the range between 0.006 and 0.009% wt, or between 0.007% wt and 0.009% wt or between 0.008% wt and 0.009% wt; or between 0.0085% wt and 0.009% wt.

In one embodiment, the steroid comprises Mometasone furoate. The Mometasone furoate may be present in the range of 0.006% wt and 0.009% wt, or between 0.007% wt and 0.009% wt or between 0.008% wt and 0.009% wt; or between 0.0085% wt and 0.009% wt. Typically, the Mometasone furoate is present at a concentration of approximately 0.0086% wt.

In another embodiment, the steroid comprises betamethasone. In this embodiment, the betamethasone is at a concentration of less than 0.01% wt. Particularly, the betamethasone may be at a concentration ranging from 0.006% wt and 0.009% wt, or between 0.007% wt and 0.009% wt or between 0.008% wt and 0.009% wt; or between 0.0085% wt and 0.009% wt. The betamethasone may be at a concentration of approximately 0.0086% wt.

In a further embodiment, the at least one antifungal agent comprises Terbinafine. Alternatively, the antifungal agent may comprise Itraconazole, Ketaconazole Ciclopirox, Clotrimazole, Econazole, Miconazole, Naftifine, Nystatin, Oxiconazole, Sertaconozole, Sulconazole or Tonaftate or a combination thereof.

The at least one anti-fungal may be present at less than 5% wt of the composition. Typically, the antifungal agent is present at less than 4% wt, or less than 3% wt. Still further, the antifungal may be at a concentration of less than 2% wt or less than 1% wt. The antifungal agent may be present in a range of from 0.5% wt to 1.5% wt.

In another embodiment, the range may be 0.6% wt to 1.4% wt; or 0.7% wt to 1.3% wt; or 0.8% wt to 1.2% wt; or 0.8% wt to 1.1% wt; or 0.85% wt to 1.0% wt; or 0.85% wt to 0.9% wt. The antifungal may be present in a concentration of 0.86% wt.

In a particular embodiment, the at least one antifungal agent comprises Terbinafine hydrochloride at a concentration range of from 0.5% wt to 1% wt of the composition. In another embodiment, said Terbinafine hydrochloride may be at a concentration ranging from 0.8% wt to 0.9% wt. In one preferred embodiment the terbinafine of the composition comprises 0.86% wt.

Other naturally occurring antifungal agents may be included in the composition including one or more of allicin, tea tree oil, citronella oil, iodine, olive leaf, orange oil, palmarosa oil, patchouli, lemon myrtle, neem seed oil, coconut oil, zinc, or selenium

The antimicrobial agent of the composition is typically Merbromine which is commonly marketed under the trade name Mercurochrome™.

The antimicrobial agent may be present in the composition at a concentration of less than 2% wt. Still further, the antimicrobial agent may be present at a concentration of less than 1% wt. In one embodiment, the composition comprises said antimicrobial agent at a concentration in the range of 0.1% wt to 1.0% wt. In a further embodiment the range of concentration of said antimicrobial agent is 0.2% wt to 0.9% wt; or 0.3% wt to 0.8% wt; or 0.4% wt to 0.6% wt; or 0.4% wt to 0.5% wt.

In one preferred embodiment the composition comprises Merbromine at a concentration of approximately 0.43% wt.

The pharmaceutically acceptable excipient of the composition may comprise any one or more of an emulsifier, emollient, solvent, or humectant.

Examples of suitable emollients include paraffinum liquidum, petrolatum, proplylene glycol, fatty acid esters, mineral oil including dimethicone, waxes including white wax, spermacetic wax, squalene, cetearyl alcohol, cetostearyl alcohol or stearyl alcohol.

Examples of suitable emulsifiers include ceteth-20, laureth-3, glyceryl stearate, polyethylene glycol or stearic acid.

Examples of suitable solvents include isopropyl alcohol, propylene glycol, butylene glycol, hexylene glycol, carbomer 934P or polyethylene glycols.

Examples of humectants include glycerin and sorbitol.

The composition may further include pH adjuster agents such as buffering agents including sodium phosphate monobasic dehydrate; acids such as phosphoric acid hydrochloric acid or bases such as sodium hydroxide.

The composition may further include one or more preservatives. Examples of suitable preservatives include benzyl alcohols including dichlorobenzyl alcohol or parabens including methyl paraben.

In another embodiment, the composition may further include purified water and hydroxypropyl cellulose.

In a still further embodiment, the composition may contain one or more antibacterial agents. For example, the composition may contain one or more antibiotics. The antibiotic may be selected from one or more of the classes that include but are not limited to penicillins, cephalosporins, carbapenems, aminoglycosides, sulfonamides, quinolones, or oxazolidinones.

The composition may also include one or more anti-viral agents. An example of an antiviral is acyclovir.

Still further, the composition may contain an antihistamine agent. The antihistamine may be selected from the group comprising piperazines, alkylamines or phenothiazines. Alternatively, an oral antihistamine may be administered concurrently with topical administering of the composition of the present invention.

The composition for topical application may comprise a cream. Alternatively, the composition may comprise an ointment. Still further, the composition may be in the form of a lotion, paste, gel, spray or powder.

The composition of the present invention may be used in a number of skin conditions. The composition has particular application in eczema. Further conditions which may be treated by the composition include but are not limited to:

Contact dermatitis;

Rashes;

Psoriasis;

Impetigo;

Fungal infections;

Bacterial skin infections;

Viral skin infection;

Yeast infections;

Trauma or injury to the skin;

Pityriasis vesicolor;

Nappy rash;

Hyperhydrosis;

Smelly armpits or feet;

Acne;

Idiopathic itchy skin;

Skin burns; or

Scarring.

### EXAMPLES

### Example 1

A composition for topical application was formulated including:

0.43% Merbromine;

0.86% Terbinafine hydrochloride; and

0.0086% Mometasone furoate.

These ingredients were mixed with a base excipient which included:

Petrolatum;

Refined mineral oil;

Cetostearyl alcohol;

Glyceryl monostearate (self emulsifying);

Squalane;

Stearic acid;

Purified water;

Dichlorobenzyl alcohol;

Macrogol cetostearyl ether;

Glyceryl monostearate 40-55;

Macrogol lauryl ether;

Methyl parahydroxybenzoate E218; and

Dimethicone

The base used in Example 1 is QV™ cream sold by Ego Pharmaceuticals.

### Case 1

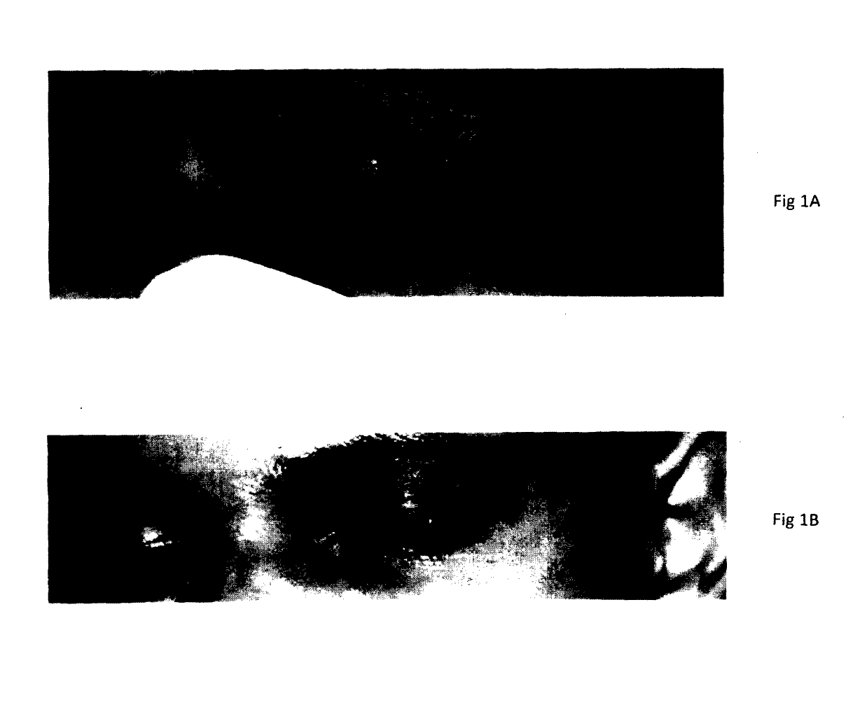

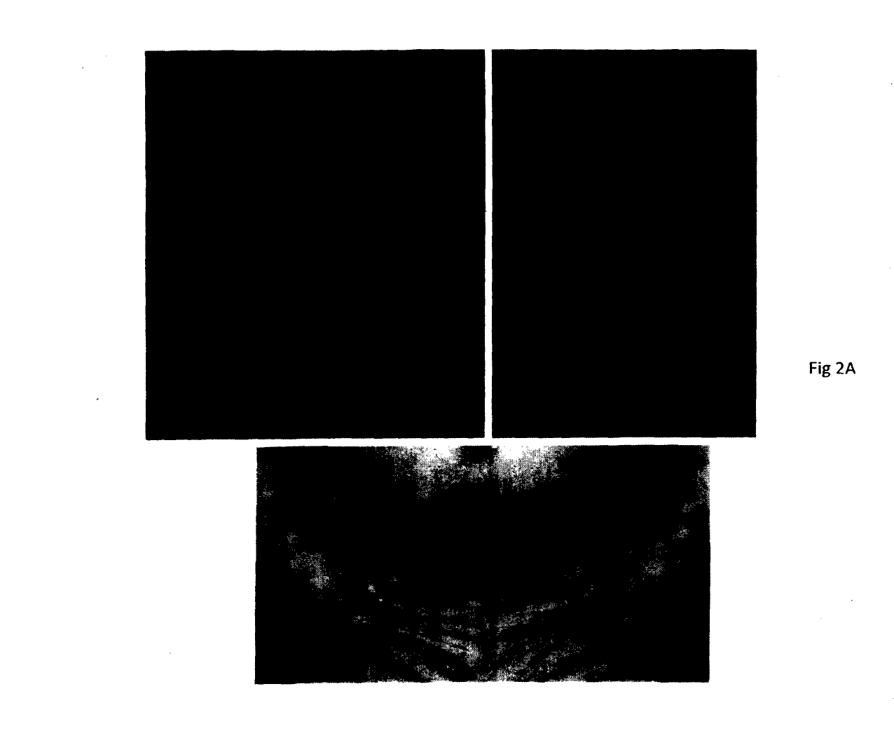

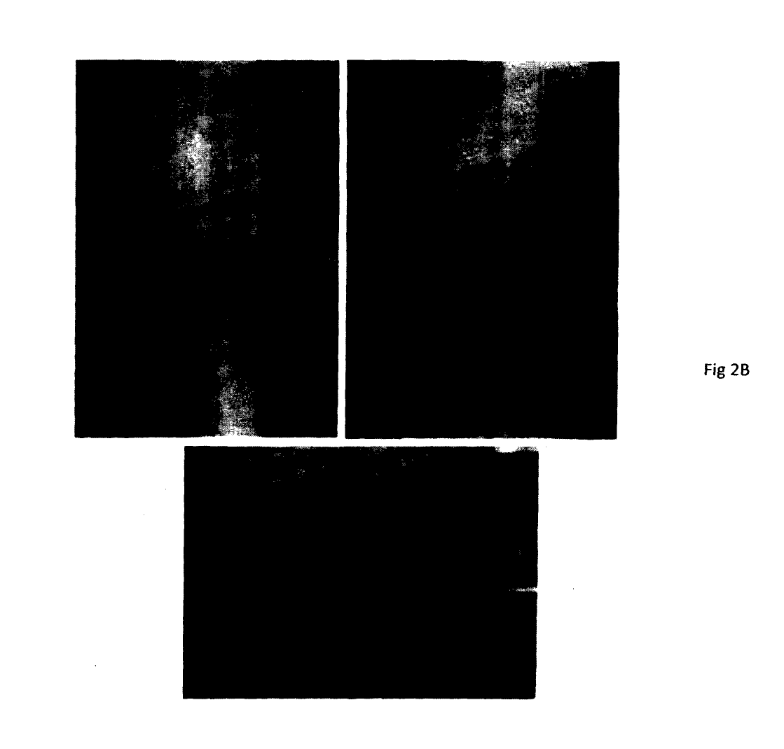

A 27 year old male with an itchy rash around the left eye for more than six months presented. Examination revealed an inflamed scaly rash with erythema and lichenification—see FIG. 1A.

He had been to two dermatologists and had previously used Terbinafine Hydrochloride 1.0% (sold under the trade name Lamisil™), Mometasone Furoate 0.1%, Hydrocosrtisone acetate 1% (sold under the trade name Sigmacort™), or Hydrocortisone (microfine) 1% w/w and clotrimazole 1% w/w (sold under the trade name Hydrozole™) with no long term improvement. A diagnosis of periorbital dermatitis with lichenification was made and treatment using the formulation of Example 1 commenced.

After 1 week, his rash had almost completely cleared (FIG. 1B). A follow up three months later confirmed no recurrence.

### Case 2

A 30 year old male with a three year history of abdominal and lower limb rash presented. The rash was occasionally itchy. Examination revealed large rash patches of 20×25 cm on the abdomen and right leg. These rash patches had distinct inflamed, erythematous active borders and scaly skin in the centres. The patient had previously trialed Griseofulvin (sold under the trade name Grisovin™), Terbinafine Hydrochloride 1.0% and Mometasone Furoate 0.1%.

A diagnosis of Tinea corpis, tinea curis was made and treatment commenced with the formulation of Example 1.

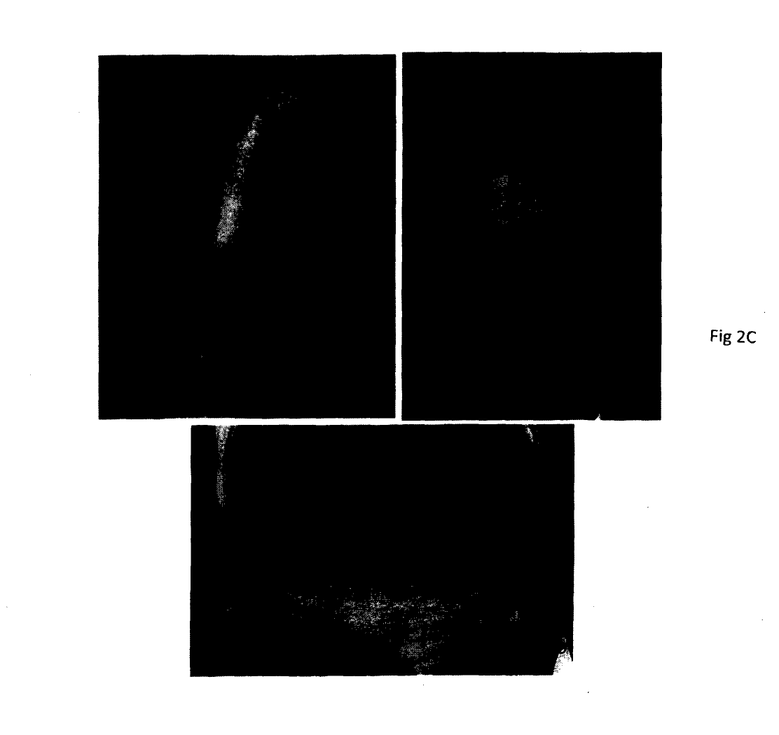

The patient reported an improvement in itchiness overnight and clinical examination showed 50% improvement after one week (see FIG. 2A before and FIG. 2B after 1 week). The rash completely disappeared after one month and nine months later showed no sign of recurrence (FIG. 2C).

### Case 3

An 11 year old boy suffering from eczema from the age of 6 months old presented. He had tried many products including Kenacomb™ (Triamcinolone acetonide, nystatin, gramicidin, neomycin); Terbinafine Hydrochloride 1.0% (Lamisil™), Methylprednisolone aceponate (sold under the trade name Advantan™); Betamethasone 0.05% (sold under the trade name Celestone™); Triamcinolone acetonide 0.5% (sold under the trade name Aristocort™) and Hydrocortisone (microfine) 1% w/w and clotrimazole 1% w/w (sold under the trade name Hydrozole™).

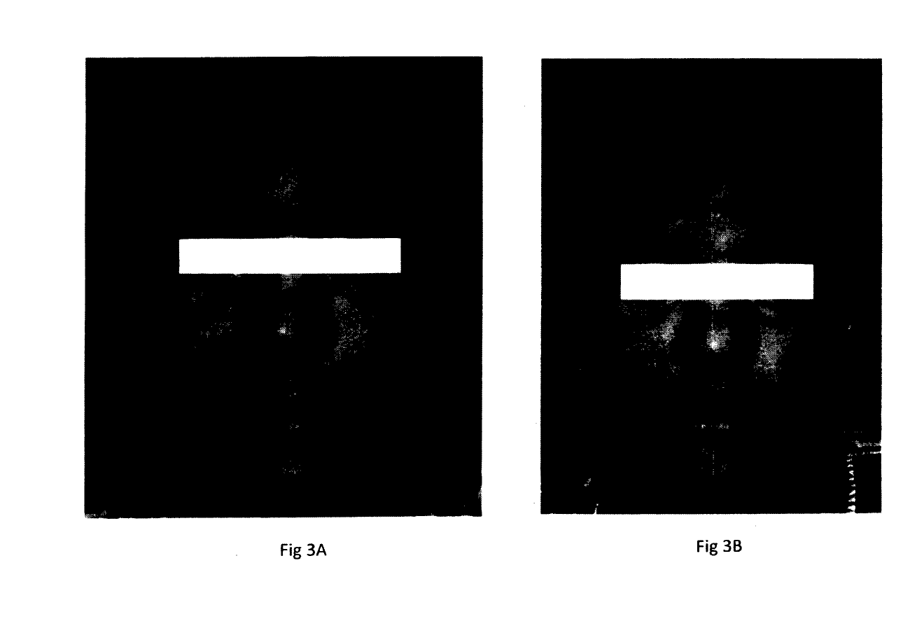

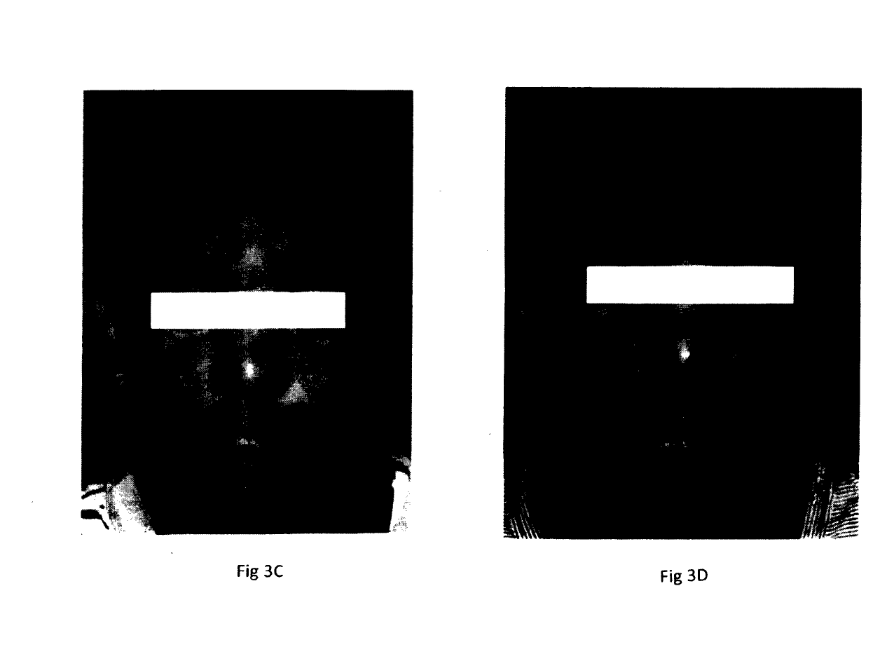

The boy suffered constant itchiness and had a generalised rash over his whole body from face to toes. The rash was diffused with no distinctive border. It showed a high degree of scaly and thickened skin, erythema of differing severity and there were extensive excoriation marks. His face was the worst affected area with severe erythema and fresh evidence of itching and scratching with open wounds, dried blood and scab formation, see FIG. 3A.

Chronic atopic dermatitis with lichenification and subclinical infection was diagnosed.

Treatment was commenced with the composition of Example 1. After 1 week, improvements were already observed (see FIG. 3B). After 3 weeks, most of the erythema had resolved and there were no fresh open wounds and excoriation had reduced significantly as can be seen in FIG. 3C. After 5 weeks the facial skin was almost recovered—see FIG. 3D.

### Case 4

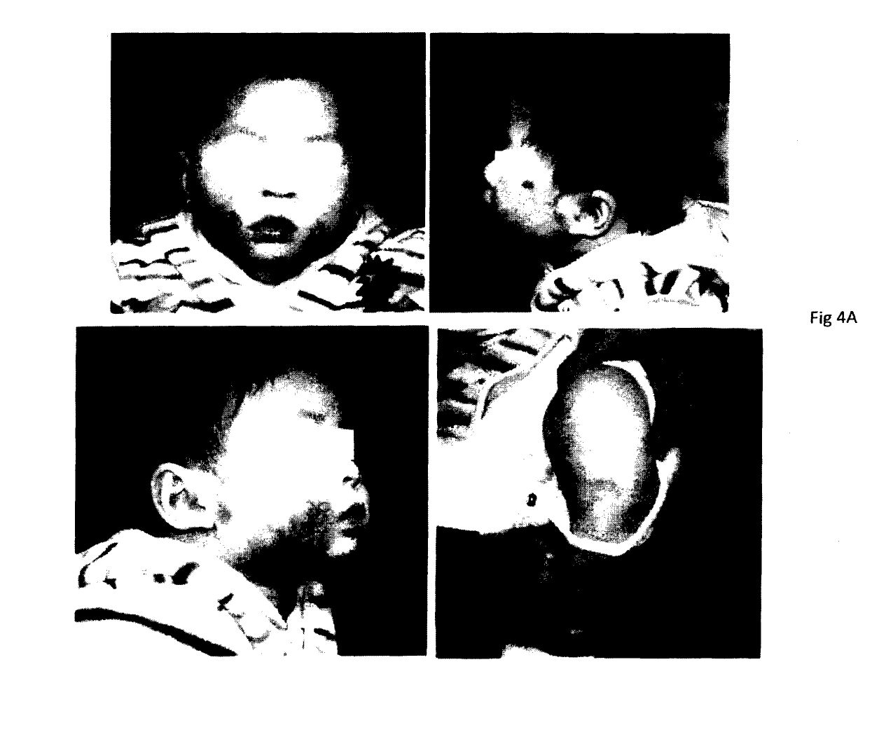

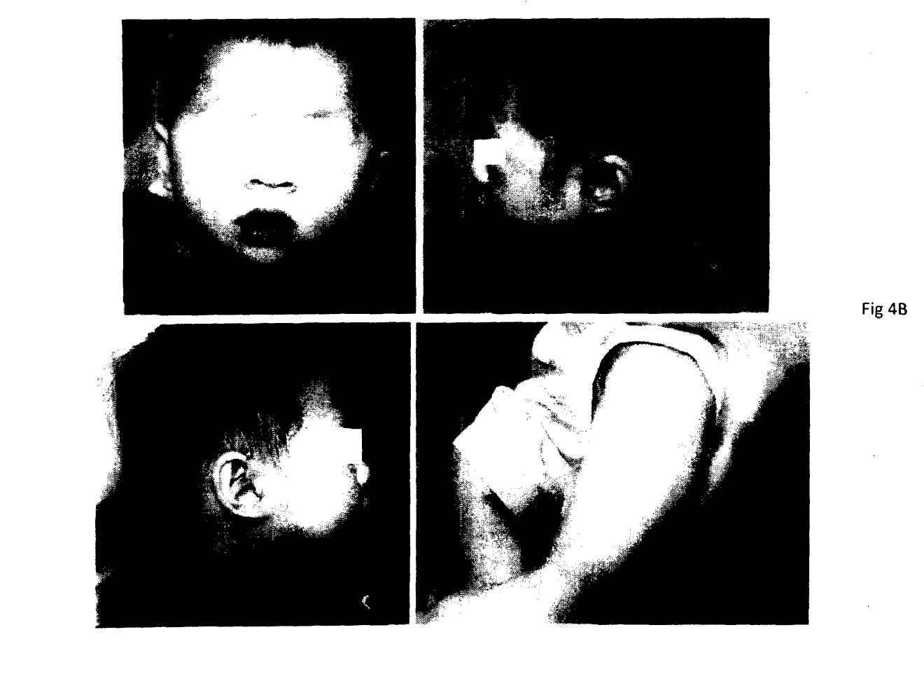

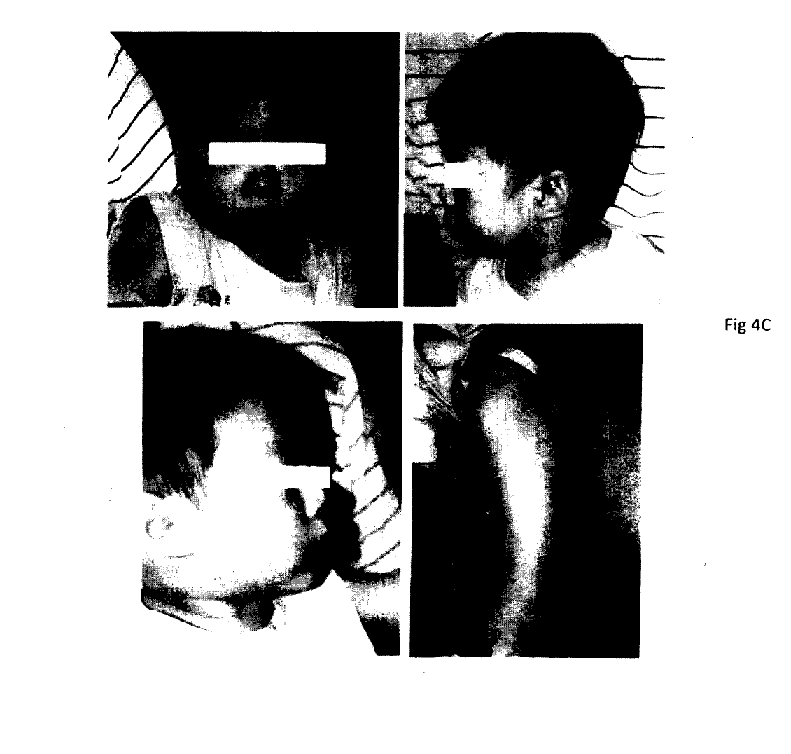

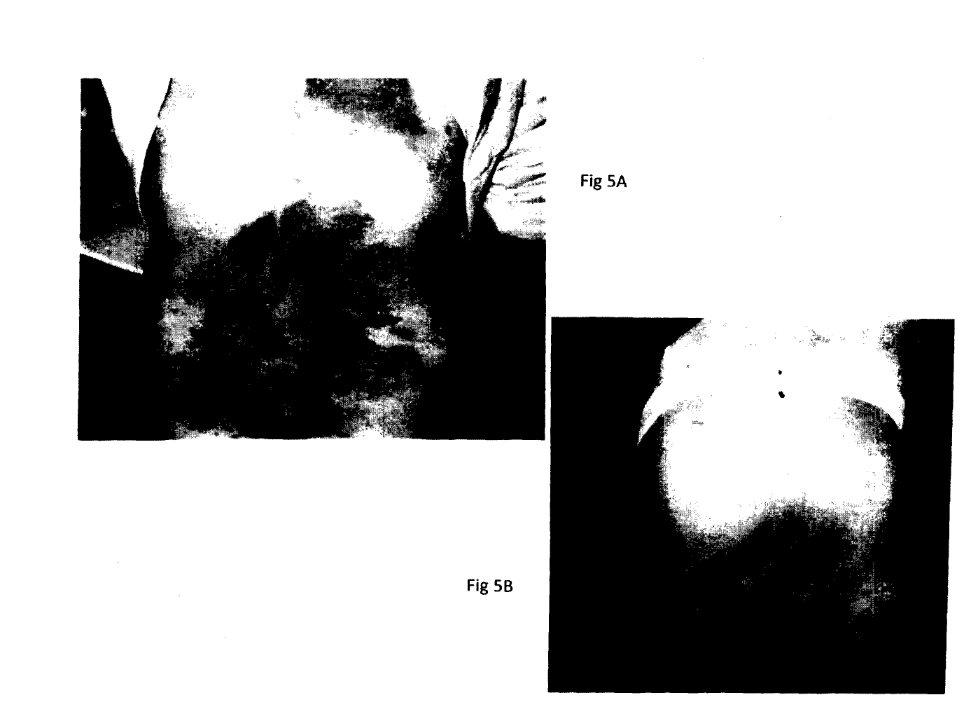

A seven month old baby boy presented with body rash and facial rash which he had had for several months. The rash on the cheeks was quite severely inflamed with crusty lesions and exudate as shown in FIG. 4A. The child had previously been treated with Mometasone Furoate 0.1% (sold under the trade name Elocon™), Hydrocortisone acetate 1% (sold under the trade name Sigmacort™), Hydrocortisone (microfine) 1% w/w and clotrimazole 1% w/w (sold under the trade name Hydrozole™) and Clotrimazole (sold under the trade name Canestan™).

A diagnosis of eczema with secondary impetigo was made.

Treatment was commenced with the composition of Example 1. After 5 weeks, the skin had improved by around 90% as shown in FIG. 4B and after 8 weeks, the skin had fully recovered (FIG. 4C). At a 9 month follow up, it was observed that there had been no re-occurrence.

### Case 5

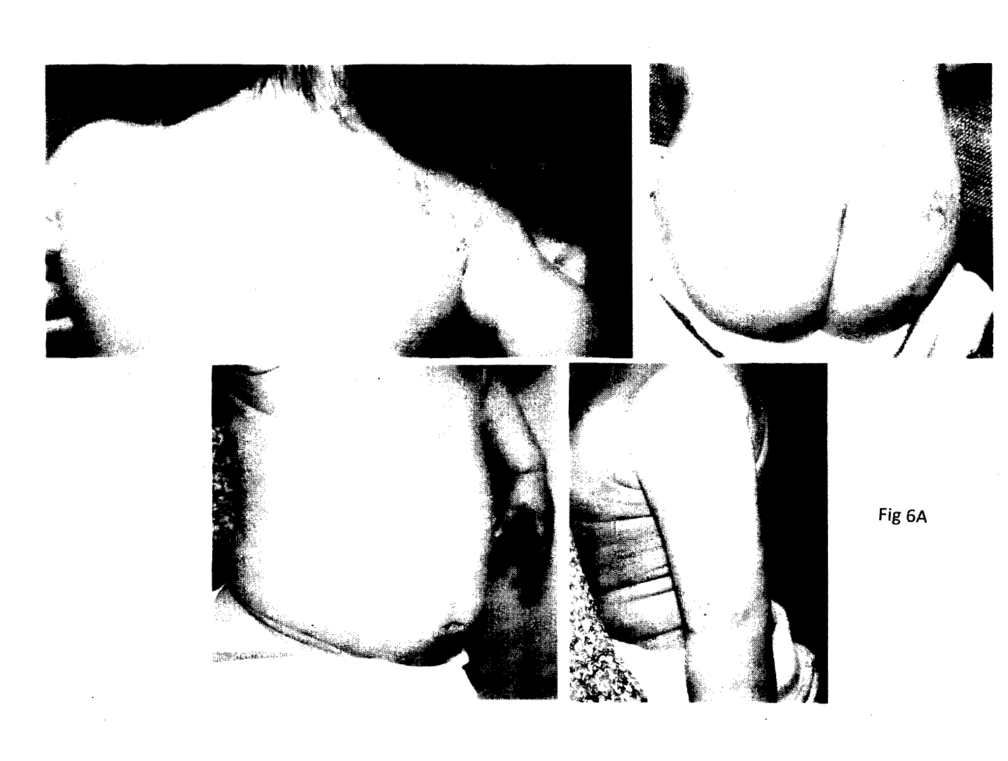

A 73 year old woman presented with a rash on her back. She had had the rash for 20 years. It was itchy and disturbed her sleep. She reported trying a number of products including Betamethasone dipropionate 0.05% (sold under the trade name Diprosone OV™), Mometasone Furoate 0.1% (Elocon™), Terbinafine Hydrochloride 1.0% (Lamisil™), Hydrocortisone (microfine) 1% w/w and clotrimazole 1% w/w (Hydrozole™). On examination, a large rash covering more than 70% of the surface area of her back was revealed. The rash was raised, inflamed and erythematous. There was extensive scaling and excoriation marks—see FIG. 5A.

A diagnosis of chronic infected dermatitis on a background of psoriasis was made.

Treatment was commenced with the composition of Example 1.

After two months the rash had completely disappeared and at a 9 month follow up there was no recurrence—see FIG. 5B.

### Case 6

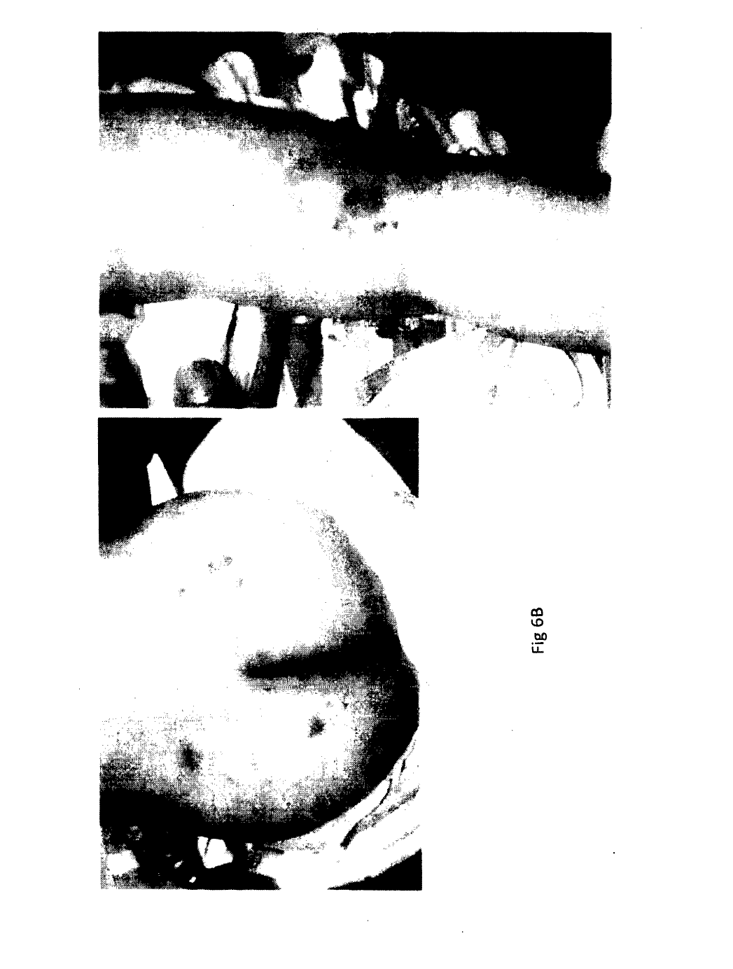

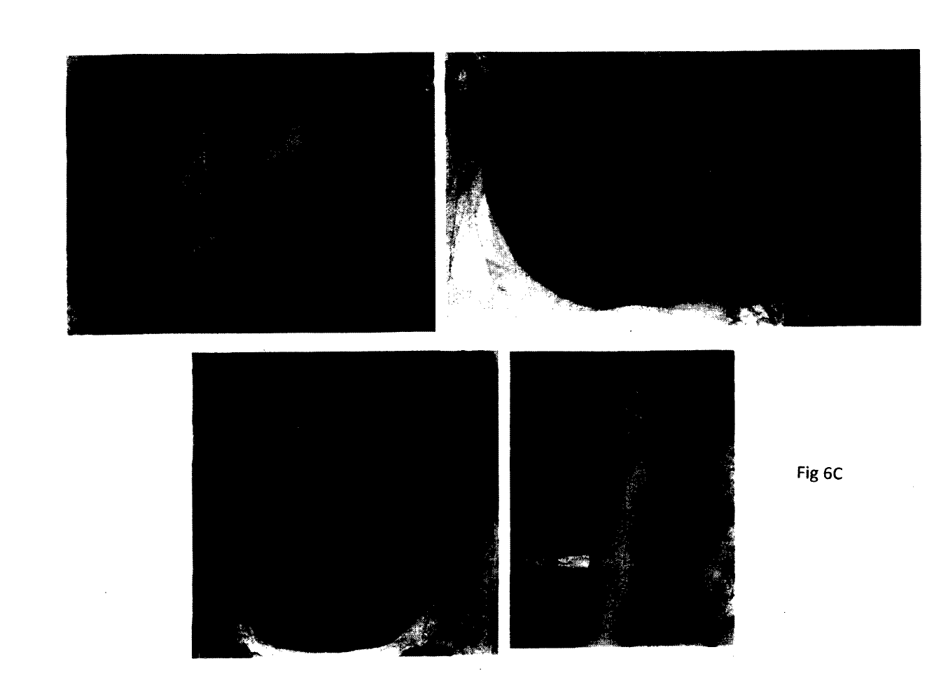

An 18 month old girl who had had eczema since birth presented. Prior treatments included Mometasone Furoate 0.1% and Terbinafine Hydrochloride 1.0%. Examination revealed a generalised rash. The rash was scaly, erythematous and inflamed. Her skin was very dry and there were numerous excoriation marks and patches of crusted skin (FIG. 6A).

A diagnosis of eczema was made.

Treatment with the composition of Example 1 was commenced.

After 1 week, approximately 50% clearance of the rash was observed (see FIG. 6B. After 7 weeks, the skin was fully recovered (FIG. 6C).

### Case 7

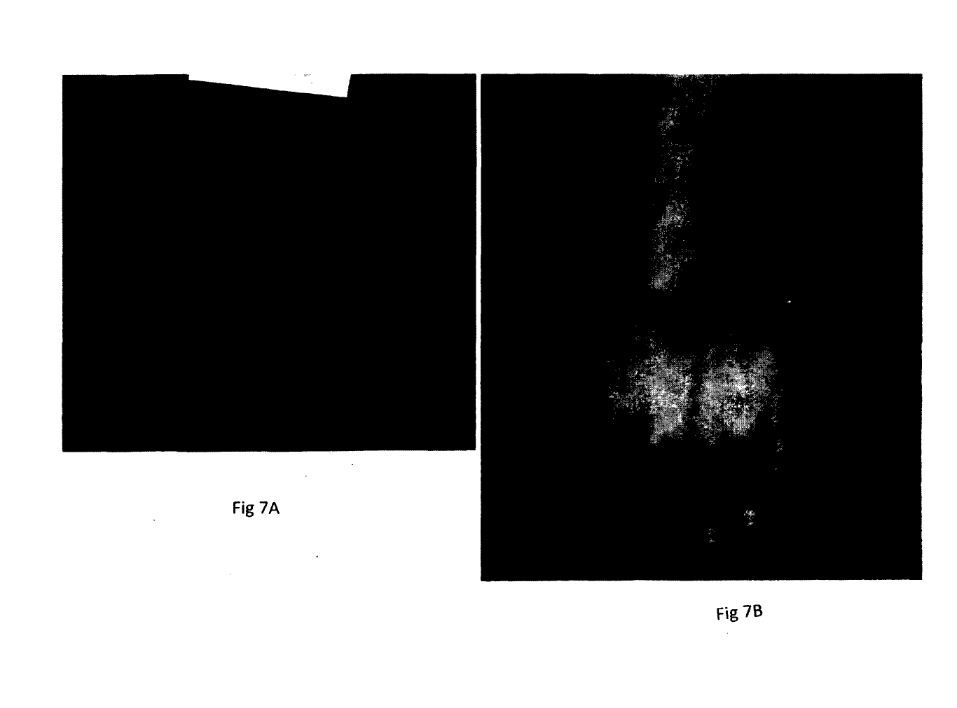

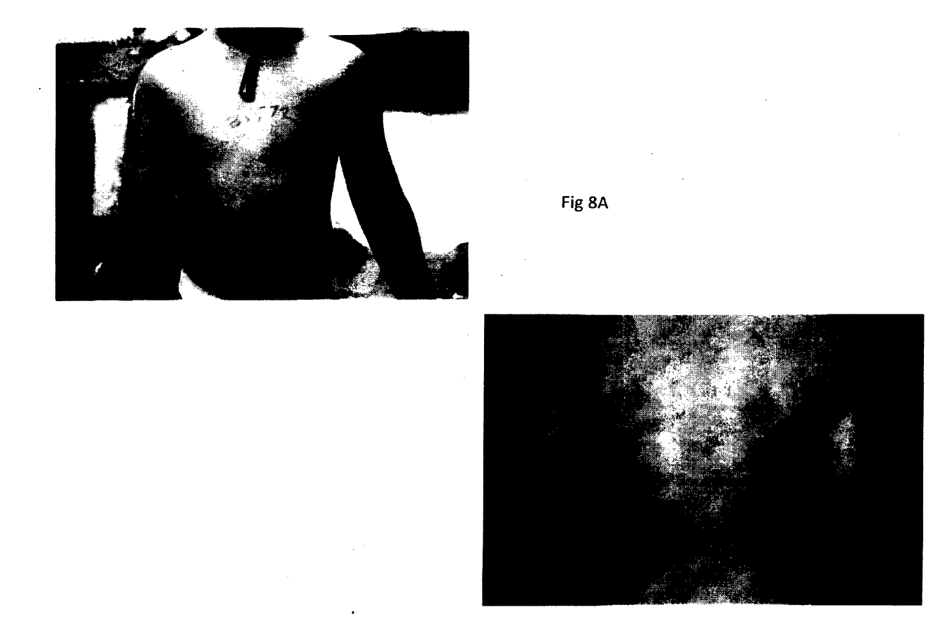

A 40 year old female with several year history of an itchy rash on the dorsum of her right foot presented (see FIG. 7A). She had previously tried many creams including various antifungal treatments, antibiotics and steroids, Particularly, she had tried Kenacomb™ (Triamcinolone acetonide, nystatin, gramicidin, neomycin); Mometasone Furoate 0.1% (Elocon™); Terbinafine Hydrochloride 1.0% (Lamisil™); Clotrimazole (sold under the trade name Canestan™) and Mupirocin (2% w/w) (sold under the trade name Bactroban™). None of the treatments gave her any long term results. Examination revealed a scaly thickened skin rash of 3×4 cm. There were areas of broken skin with scattered scab formation.

A diagnosis of chronic dermatitis with lichenification was made and treatment commenced with the composition of Example 1.

The patient reported an almost instant relief of itchiness. In less than one week the rash had disappeared (FIG. 7B). At an 11 month follow up, there were no signs of recurrence.

### Case 8

A nine year old girl presented with a generalised body rash which she had had for a few years. She had tried many products including Terbinafine Hydrochloride 1.0% (Lamisil™), Betamethasone dipropionate 0.05% (sold under the trade name Diprosone OV™), Hydrocosrtisone acetate 1% (sold under the trade name Sigmacort™), Clotrimazole (sold under the trade name Canestan™); Methylprednisolone aceponate (sold under the trade name Advantan™) with no permanent improvement.

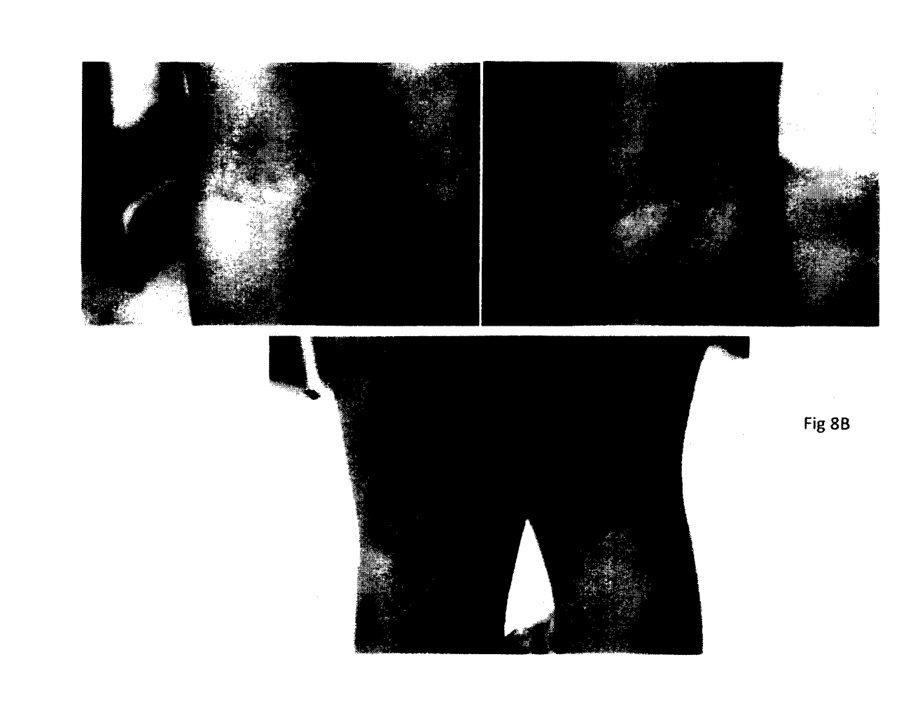

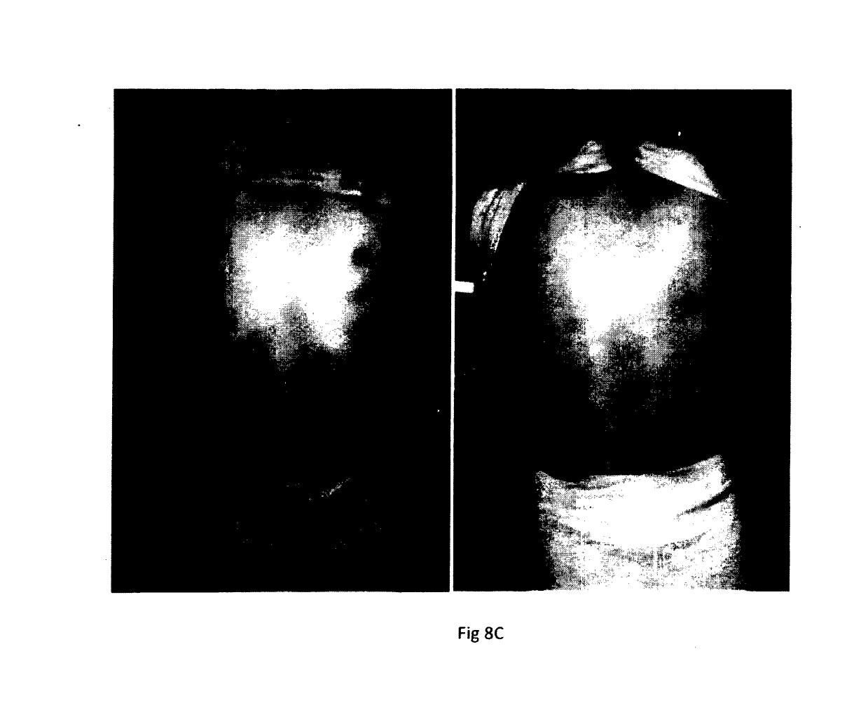

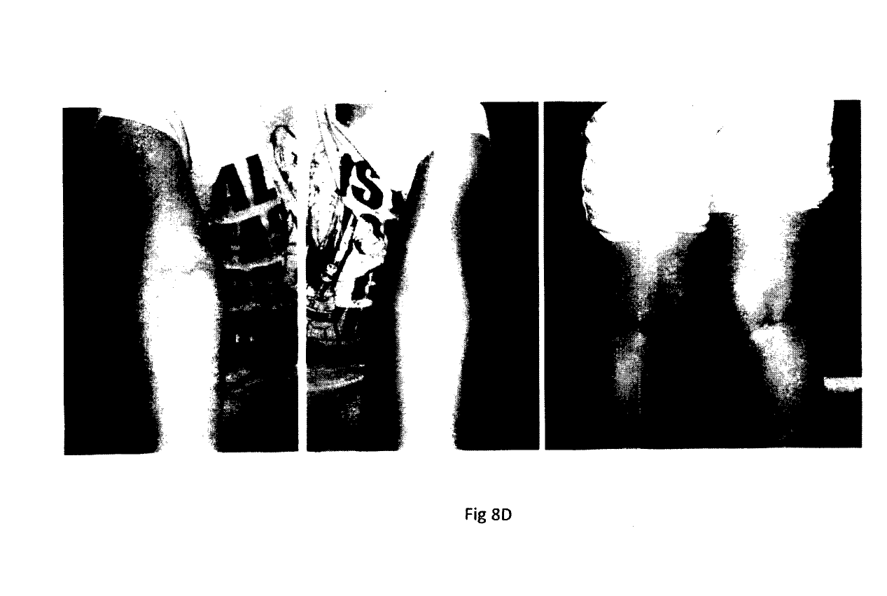

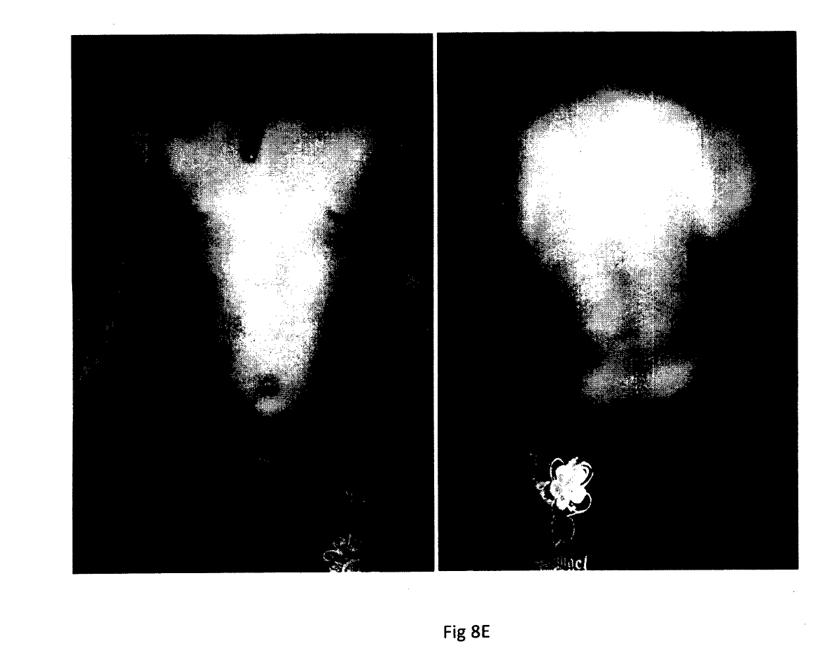

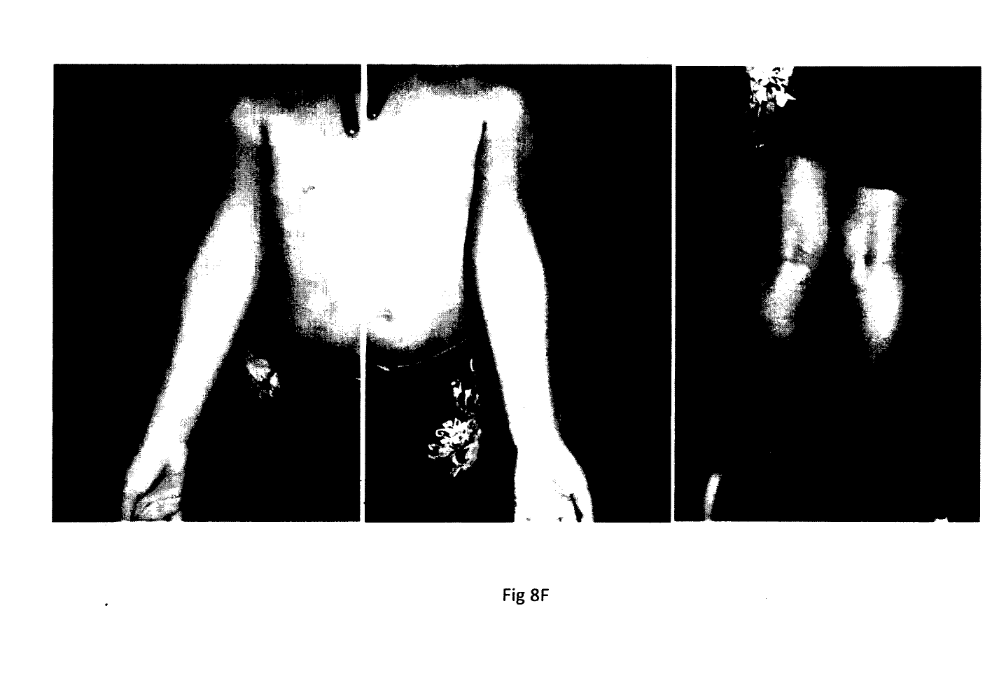

Examination revealed typical eczematous rash spread all over the body which was more severe on cubital and popliteal fossa. The rash was scaly, flaky, had a diffused border, erythematous background and there were excessive excoriation marks and a thickening of the skin (see FIGS. 8A and B). A diagnosis of chronic atopic dermatitis was made.

Treatment was commenced with the composition of Example 1 and in 4 months the skin had largely cleared (FIGS. 8C and D). By 6 months the skin had fully cleared (FIGS. 8E and F). At a 7 month follow up there was no recurrence. An 11 month follow up by phone confirmed no recurrence.

### Further Studies

50 cases of chronic eczema (more than 5 months duration) were selected for an 11 month follow up. These patients were ranged from 5 months to 12 years old. They must have been on one of a topical corticosteroid, an antifungal cream or combination for more than 1 month. They must have had no results or limited improvement from the conventional therapy (including occlusive treatment with topical steroid).

The results and data collected from the patients were as follows:

### 1. Symptom Relief

A). 3 patients achieved immediate symptom relief (6%)

B). 30% of the patients (including patients in group A) reported symptom relief after 24 hours.

C). 88% of the patients (including patients in group A and B) reported symptom relief after 1 week.

D). 100% of the patients reported symptom relief after 3 weeks.

### 2. Clinical Clearance

A) 32% (16 patients) of the patient achieved total clinical clearance after 4 weeks.

B) 70% (35 patients) of the patients achieved total clinical clearance after 3 months

C) 86% (43 patients) of the patient achieved total clinical clearance after 6 months.

### 3. Relapse

An 11 month follow up was conducted on 43 out of the 50 patients. 9.3% had relapsed (4 out of 43). 3 out of 4 (75%) of the relapsed patients admitted poor compliance (stopped treatment too soon).

The composition of Example 1 promotes healthy skin formation as evidenced in the above examples. All three of the main ingredients in this embodiment, being an antifungal, a steroid and an antimicrobial agent are included at well below the considered therapeutic range. For example, the anti inflammatory ingredient Mometasone furoate is at 0.0086% concentration whereas the same agent on the market is at 0.1% concentration. Terbinafine hydrochloride is at 0.86% wt whereas on the market, the therapeutic dose is 1%. The Merbromine is at a concentration of 0.43% wt whereas the market concentration is 2% in Australia although concentrations of 1% are available in other regions.

In this embodiment, all three ingredients are well below the expected therapeutic concentration for each individual ingredient and it has been found that they unexpectedly together produced the clearly successful outcomes in patients as hereinbefore described.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A topical composition for the treatment of a skin condition, said composition comprising:
less than 0.01% wt of at least one anti-inflammatory agent;
at least one antifungal agent;
an antimicrobial agent of the general formula: 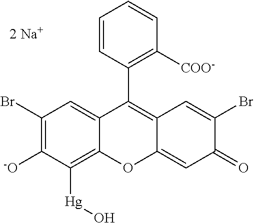
and a pharmaceutically acceptable excipient.

2. The topical composition of claim 1 wherein the anti-inflammatory agent is a topical steroid.

3. The topical composition of claim 2 wherein the steroid is selected from the group including Hydrocortisone, Alclometasone dipropionate, Triamcinolone acetonide, Fluocinolone acetonide, Fluticasone propionate, Hydrocortisone valerate, Hydrocortisone butyrate, Flurandrenolide, Mometasone furoate, Betamethasone, Fluocinonide, Halcinonide, Amcinonide, Desoximetasone, Clobetasol propionate, Halobetasol proprionate, Diflorasone diacetate, Diflucortolone valerate, Hydrocortisone 17-butyrate, Methylprednisolone aceponate or Clobetasone butyrate or a combination thereof.

4. The topical composition of claim 1 wherein the anti-inflammatory agent comprises Mometasone furoate.

5. The topical composition of claim 1 wherein the anti-inflammatory agent comprises Betamethasone.

6. The topical composition of claim 1 wherein the anti-inflammatory agent is at a concentration of less than 0.009% wt.

7. The topical composition of claims 1 wherein the anti-inflammatory agent is at a concentration of between 0.008% wt and 0.009% wt.

8. The topical composition of claim 4 wherein the Mometasone furoate is at a concentration of approximately 0.0086% wt.

9. The topical composition of claim 1 wherein the at least one antifungal agent is selected from the group including Terbinafine hydrochloride, Ciclopirox, Clotrimazole, Econazole, Miconazole, Naftifine, Nystatin, Oxiconazole, Sertaconozole, Sulconazole or Tonaftate, Itraconazole, Ketaconazole or a combination thereof.

10. The topical composition of claim 1 wherein the at least one antifungal agent comprises Terbinafine hydrochloride.

11. The topical composition of claim 1 wherein the at least one antifungal agent is present at less than 5% wt.

12. The topical composition of claim 1 wherein the at least one antifungal agent is present at less than 1% wt.

13. The topical composition of claim 10 wherein the Terbinafine hydrochloride is at a concentration of between 0.5% wt and 1.0% wt.

14. The topical composition of claim 10 wherein the Terbinafine hydrochloride is at a concentration of approximately 0.86% wt.

15. The topical composition of claim 1 further including any one of, or a combination of, naturally occurring antifungal agents selected from allicin, tea tree oil, citronella oil, iodine, olive leaf, orange oil, palmarosa oil, patchouli, lemon myrtle, neem seed oil, coconut oil, zinc, or selenium.

16. The topical composition of claim 1 wherein said antimicrobial agent comprises Merbromine.

17. The topical composition of claim 16 wherein said Merbromine is at a concentration of less than 2% wt.

18. The topical composition of claim 16 wherein the Merbromine is at a concentration in the range of 0.1% wt to 1.0% wt

19. The topical composition of claim 16 wherein the Merbromine is at a concentration of approximately 0.43% wt.

20. The topical composition of claim 1 wherein the pharmaceutically acceptable excipient of the composition comprises any one or more of an emulsifier, emollient, solvent, or humectant.

21. The topical composition of claim 20 wherein said emollient is selected from one or a combination of the group including paraffinum liquidum, petrolatum, proplylene glycol, fatty acid esters, mineral oil including dimethicone, waxes including white wax, spermacetic wax, squalene, cetearyl alcohol, cetostearyl alcohol or stearyl alcohol.

22. The topical composition of claim 20 wherein said emulsifier includes one or more of ceteth-20, laureth-3, glyceryl stearate, polyethylene glycol or stearic acid; or a combination thereof.

23. The topical composition of claim 20 wherein said solvent is selected from the group including isopropyl alcohol, propylene glycol, butylene glycol, hexylene glycol, carbomer 934P or polyethylene glycols or a combination thereof.

24. The topical composition of claim 20 wherein the humectant includes glycerin or sorbitol.

25. The topical composition of claim 1 further including a pH adjuster including sodium phosphate monobasic dehydrate; phosphoric acid, hydrochloric acid or sodium hydroxide.

26. The topical composition of claim 1 the form of a cream.

27. The topical composition of claim 1 in the form of an ointment, a lotion, a paste, a gel, a spray or a powder.

28. The composition of claim 1 for use in the treatment of eczema.

29. The composition of claim 1 for use in the treatment of contact dermatitis.

30. The composition of claim 1 for use in the treatment of rashes.

31. The composition of claim 1 for use in the treatment of psoriasis.

32. The composition of claim 1 for use in the treatment of impetigo.

33. The composition of claim 1 for use in the treatment of any one of fungal infections; bacterial skin infections; viral skin infection; yeast infections;
trauma or injury to the skin; pityriasis vesicolor; nappy rash; hyperhydrosis; smelly armpits; acne;
idiopathic itchy skin; skin burns; or scarring.

34. A topical composition for the treatment of a skin condition, said composition comprising:
at least one corticosteroid;
at least one antifungal agent; and
Merbromine.
